(19) United States
(12) Patent Application Publication
(10) Pub. No.: US 2012/0123392 A1
(43) Pub. Date: May 17, 2012

(54) LUER CONNECTOR

(75) Inventors: Robert J. McKinnon, Highlands Ranch, CO (US); James Dale Bickley, Tucson, AZ (US)

(73) Assignee: WestMed, Inc., Greenwood Village, CO (US)

(21) Appl. No.: 13/297,128

(22) Filed: Nov. 15, 2011

Related U.S. Application Data
(60) Provisional application No. 61/414,323, filed on Nov. 16, 2010.

(51) Int. Cl. A61M 25/16

(52) U.S. Cl. 604/533

(57) ABSTRACT

A luer connector is provided having a first member that includes an end face with a reflective material that does not cover the entire end face wherein the first member is selectively rotatable to align the reflective portion with sensors of the medical instrument. The first member is adapted to freely rotate within a second member that is used to connect the first member to the medical instrument.

This application claims the benefit of U.S. Provisional Patent Application Ser. No. 61/414,323, filed Nov. 16, 2010, the entire disclosure of which is incorporated by reference herein.

### FIELD OF THE INVENTION

Embodiments of the present invention generally relate to sensing the presence and verifying the identity of a tube connector. More specifically, a tube connector is provided that will be accepted by electro-optical sensors of a medical testing instrument, thereby allowing the instrument to perform its intended function.

### BACKGROUND OF THE INVENTION

Capnographs are medical instruments for analyzing exhaled air. Capnographs include a breathing tube, also called a sampling line having a first end connected to an air passageway of a respirator or to a cannula. A second end of the tube comprises a connector that mates with a complimentary connector on the medical instrument. In some situations, the connectors are unique such that the instrument will only function if the connector associated with the second end of the tube is of a specific type and manufacture. Tube assemblies may also include a filter or other means for removing moisture and mucous. Alternatively, a filter may be built into the medical instrument or supplied separately, to be connected to the tube. Tube assemblies are usually disposable and replaced after each test, which lead to great expense.

Luer connectors are commonly used in the medical industry for connecting small tubes and for attaching tubes to testing instruments. For example, luers are used to connect needles to syringes, lines for sampling blood, IVs, to bags, air/gas sampling lines, etc. Luer connectors are male or female and can be slip fit or locking, which are defined in ISO standards BS EN 1707 and BS EN 20594-1, respectively. Because luer connectors are standardized throughout the medical industry, i.e., a particular medical instrument may accept tube assemblies of various manufacturers, medical instrument manufacturers generally have no control over the type of tube that is used with their instruments.

To ensure optimal functioning of the instrument, as well as for commercial reasons, instrument manufacturers often wish to exert control over the types of tubes and connectors used with their instrument. In particular, medical instrument manufacturers often want to dictate that the instrument will only function when certain classes of tube assemblies and/or connectors are used, which is helpful when implemented for patient safety reasons to ensure the right equipment is being used for the right purpose. However, some manufacturers implement feedback systems solely for monetary gain—to require purchasers of medical instrumentation to also purchase tubing and connectors from the same manufacturer. The latter reasoning can be harmful to the medical industry as it inhibits competition and often times forces hospitals to purchase tubing and other peripheral equipment at a higher cost, which is passed on to the patient, their insurance company, and/or the federal or state government. One way medical equipment manufacturers assure that the proper equipment is being used is to employ a unique interlocking connector arrangement between the tube and the instrument. Such arrangements are often incompatible with standard connector shapes. Further, some locking arrangements require the operator to perform connecting steps and motions that may be cumbersome or bothersome to the instrument operator, technician, or healthcare professional.

A less intrusive way of accomplishing a manufacturer's wishes is to provide a system wherein the correct tube assembly is identified by the instrument. If the correct connector is sensed, operation of the instrument is enabled. A benefit of such an arrangement is that the instrument is prevented from operation when no tube is connected, when a correct tube is improperly connected or when an unauthorized tube assembly is connected, thus potentially preventing damage to sensitive instrument parts, avoiding incorrect readings, and possibly preventing patient injury. Yet another purpose may be served by such a system, namely identifying the tube assembly class to enable the instrument to operate differently to accommodate the identified particular connector class.

Several types of methods for identifying connectors are known. One type is electro-mechanical, whereby one half of the connector employs one or more protrusions or notches on its face that engage appropriately placed levers or switches on the other connector half that activate instrument-initiating microswitches. This type of instrument initiation is impractical because the connecting portions are small. Another identification method is primarily electrical, whereby the connector employs one or more conductive paths on its end face that complete a circuit when engaged with contacts on the instrument. Again, this type of identification method is impractical as the connectors are generally used in damp environments, which will often trigger unintended instrument initiation or short circuit. For example, U.S. Pat. No. 6,437,316 to Colman et al. (“Colman”) describes a device that uses a male lure lock integrated into a gas analyzer and a female luer (locking or slip) that is attached to the tubing that goes to the patient. The male luer of the machine has two fiber optic cables, one that extends from the end face of the luer to an infrared LED and one that is interconnected to a photo detector. The end portion of the female luer is provided with a reflective material. When the female luer is mated with the male luer, the light from the infrared LED passed through a fiber optic cable, is reflected off the end face of the female luer and is directed back through a second fiber optic cable to the detector which indicates to the machine that an authorized connector is present and that the connector and tubing are properly attached, and therefore is an authorized connector. In this device disclosed by Coleman, the entire end face of the female luer portion must be coated with the reflective material. This allows the female luer to be connected and recognized by the instrument in any orientation. That is, if less than 100% of the end face is coated with the reflective material, proper orientation of the reflective material cannot be ensured and the instrument may not activate.

One of the problems with a standard luer lock connector is that twisting the connector also twists to the associated line. To resolve this issue, male luer locks sometimes employ a floating nut that allows the luer connector to be engaged and then tightened to lock the luer into position without twisting the tube. This, however, is normally not practical with respect to female luer locks as the space in which they are inserted is small such that a floating nut cannot be accommodated.

There is thus a widely recognized need for, and it would be highly advantageous to provide, a fluid analyzing system that includes the capability of determining that a tube assembly has been properly connected to the instrument and that the tube is of a certain class. Such capability must be compatible with existing varieties of connectors compatible with a medical environment, reliable, and preferably inexpensive and simple to manufacture and use.

### SUMMARY OF THE INVENTION

It is one aspect of embodiments of the present invention to provide a luer connector that is used to join a tube to a medical instrument. In one specific embodiment, a luer lock connector is provided wherein a male luer connector, which is associated with a medical instrument, is interconnected to a female luer connector, which is associated with a tube. The female luer connector employs threads that mate with threads in the male luer connector. The mating connectors are engaged and tightened until the luer connections seal. As one of skill in the art will appreciate, the threads only prevent the luer adapter from disengaging and have no part in providing a sealed fluid path.

It is another aspect of embodiments of the present invention to provide a reflective pattern on a luer connector end face that provides feedback so that the medical instrument can verify that the proper luer connector is used and properly attached. Because connectors are used for a variety of different purposes and with a variety of fluids such as gases, blood, and other fluids, connecting the wrong equipment to the wrong tube or line is a potential concern. Misconnections can cause damage to equipment and/or perhaps injury to the patient. For example, a misconnection of an arterial catheter to a CO2 monitor can fill the CO2 monitor with blood causing equipment failure. More importantly, however, if an IV line were to be connected to an air pathway, liquids may be transferred into the lungs of a patient causing pneumonia or worse. To address this issue, some medical instruments that confirm that it is interconnected to the correct tube before the instrument becomes functional. In the examples above, if the arterial catheter were connected to the CO2 monitor the monitor would read the reflective pattern and alarm would sound to inform the user of the misconnection and, in addition, the instrument may be built with a feature that disables the functioning of the unit until the proper connector is used.

More specifically, often medical instruments include an optical circuit that must be completed for the medical instrument to function. The medical instrument may have several sets of fiber optic cables that correspond with points on the female luer connector end face. One contemplated medical instrument checks the areas of the female luer connector that should have reflectance and areas that should not have reflectance to determine if the correct female connector is being used. The patterns can be much more complex than what is shown in the drawings provided below, giving a large number of possible combinations of areas of reflectance and areas of no reflectance.

Further, depending on the location of the medical device's medical sensors and the nature of the marking on the female luer end face, the rotational orientation the female luer connector relative to the mating connector of the medical instrument may be critical. For example, in one embodiment, the end face of the female luer is coated with reflective material that preferably spans about 180 degrees. Thus, to ensure proper rotational alignment, the medical instrument may include an engraved line or other marking indicating of the location of an optical pathway. Similarly, a portion of the female luer connector would have an orientation indicator that is matched with that of the medical instrument. In one embodiment, the female luer possesses a protrusion or fin that is aligned with the mark provided on the medical instrument to correctly orient the female luer connector. Only when the female luer pattern on the connector is oriented in a predetermined fashion relative to the orientation mark will the medical instrument function. Thus, the contemplated connection scheme requires more human interaction to ensure the correct connectors are being used and that the connectors are properly oriented.

It is another aspect of embodiments of the present invention to provide a female luer connector assembly comprising: a first member having a first end with a flange extending therefrom, the flange having an end face, and a second end with a protrusion extending therefrom, the end face having a reflective material on less than the entire surface area thereof; and a second member having a first threaded end which is adapted to be received within mating threads of a medical instrument, and a second end having at least one protruding tube, wherein the second member operably positioned around the first member and freely rotatable relative thereto; the second member having a longitudinal slot along a surface thereof for receiving the protrusion.

It is still yet another aspect of embodiments of the present invention luer connector assembly comprising: a first member having a first end with an end face, and a second end; a reflective material on less than the entire surface area of the end face; and a second member positioned around the first member and freely rotatable relative thereto, the second member having a first end, which is adapted to be received within a medical instrument.

It is a further aspect of embodiments of the present invention to provide a connector for selective interconnection to a medical instrument, which has a male interface with a transmitter and a receiver, comprising: a first member having a first end, a flange having an end face, and a second end; a reflective material positioned on less than the entire surface area of the end face; a second member having a first end that is interconnected to the medical instrument; wherein the second member is positioned around the first member and freely rotatable relative thereto; and wherein the transmitter emits a signal that reflects off the reflective surface to be received by the receiver to indicate that the connector is suited for the medical instrument

The Summary of the Invention is neither intended nor should it be construed as being representative of the full extent and scope of the present invention. Moreover, references made herein to “the present invention” or aspects thereof should be understood to mean certain embodiments of the present invention and should not necessarily be construed as limiting all embodiments to a particular description. The present invention is set forth in various levels of detail in the Summary of the Invention as well as in the attached drawings and the Detailed Description of the Invention and no limitation as to the scope of the present invention is intended by either the inclusion or non-inclusion of elements, components, etc. in this Summary of the Invention. Additional aspects of the present invention will become more readily apparent from the Detail Description, particularly when taken together with the drawings.

### BRIEF DESCRIPTION OF THE DRAWINGS

The accompanying drawings, which are incorporated in and constitute a part of the specification, illustrate embodiments of the invention and together with the general description of the invention given above and the detailed description of the drawings given below, serve to explain the principles of these inventions.

FIG. 1 is a perspective view of a female luer connector;

FIG. 2 is a perspective view of a floating nut;

FIG. 3 is a perspective view of a female luer connector assembly of one embodiment of the present invention;

FIG. 4 is a cross-sectional view showing the female luer and floating nut before interconnection to a male luer connector;

FIG. 5 is another perspective cross-sectional view showing the female luer connector assembly before interconnection to the male luer connector, which includes a light transmitter and associated sensor;

FIG. 6 is a cross-sectional view showing the female luer connector assembly interconnected to the male luer connector; and

FIG. 7 is another cross-sectional view showing the female luer connector assembly interconnected to the male luer connector which includes a light transmitter and associated sensor.

It should be understood that the drawings are not necessarily to scale. In certain instances, details that are not necessary for an understanding of the invention or that render other details difficult to perceive may have been omitted. It should be understood, of course, that the invention is not necessarily limited to the particular embodiments illustrated herein.

### DETAILED DESCRIPTION

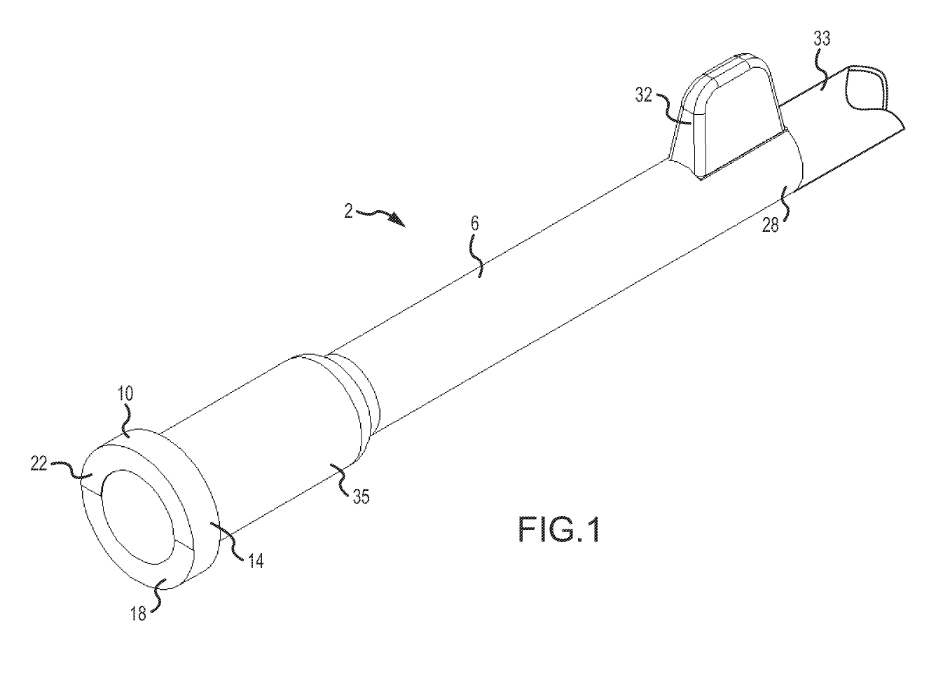

FIG. 1 shows a female luer connector 2 that comprises a cylindrical hollow body 6 having an annular flange 10 disposed at a first end 14 that is designed to interface with a male luer connector of a medical instrument, such as a capnograph. A portion of a face 18 of the annular flange 10 is coated with a reflective material 22 that at least partially covers approximately one-quarter to one-half of the face 18. It should be understood that the reflective material is intended to orient with sensors associated with a medical instrument and, therefore, may not be continuous, but may be found on the annular flange in any appropriate pattern. A second end 28 of the female luer 2 includes an upstanding flange or fin-like portion 32. The second end 28 interconnects to an oxygen tube 33, for example, that extends to a patient. The hollow body 6 of the female luer 2 may comprise an increased diameter portion 35 with an increased outer diameter.

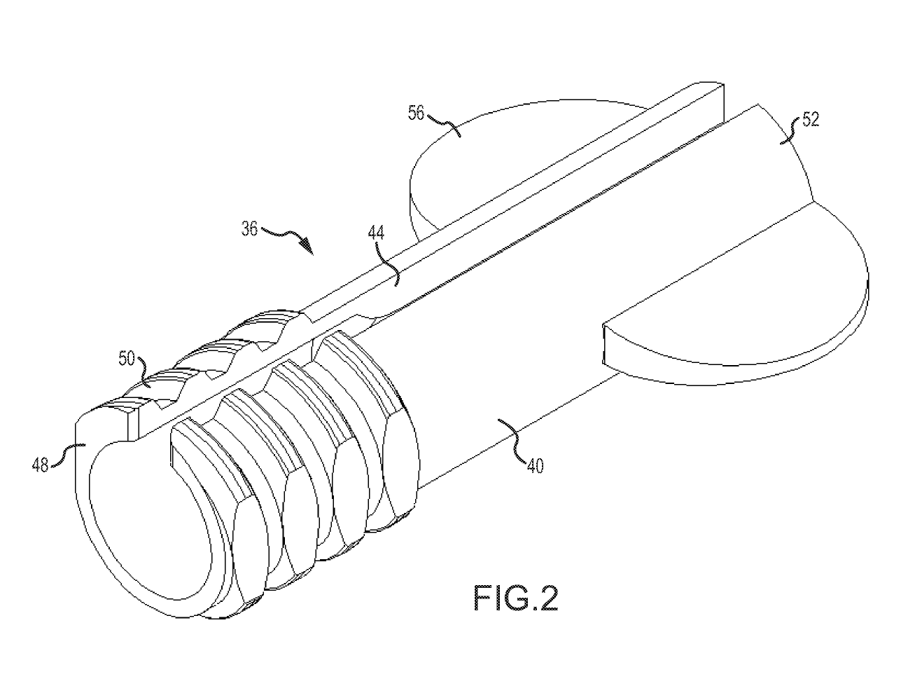

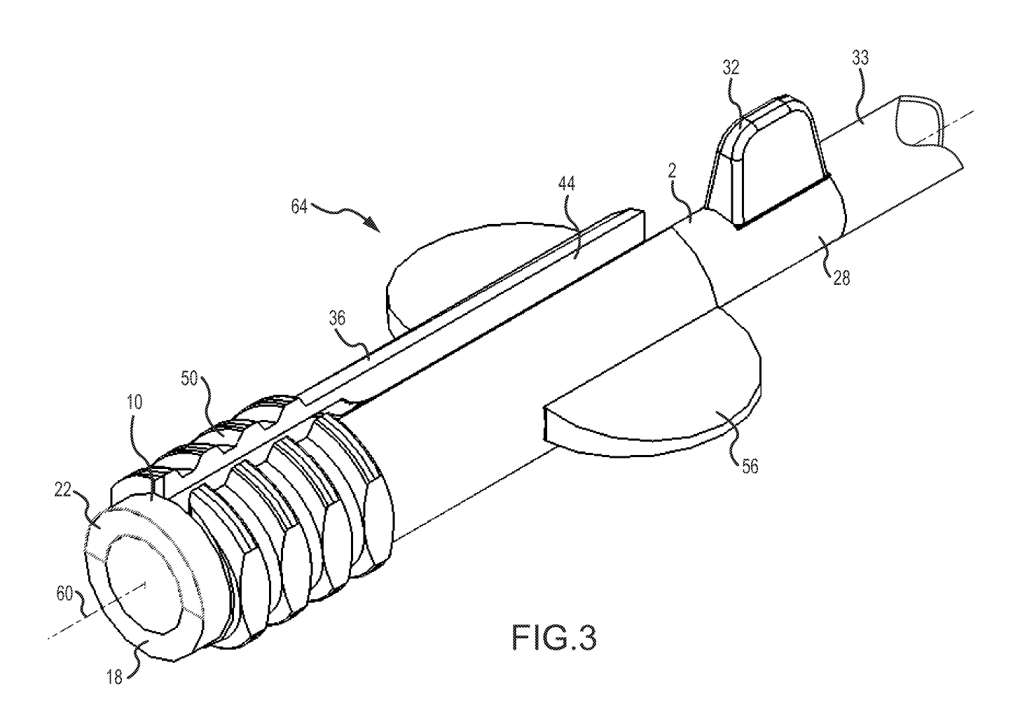

FIG. 2 shows a floating nut 36 that is generally comprised of a tube 40 with a threaded end. The tube includes a slot 44 that extends the entire length of the tube 40. A first end 48 of the tube 40 is threaded on an exterior surface and a second end 52 includes a pair of outwardly extending tubes or wings 56. The combination of the floating nut 36 and the female luer connector form a female luer connector assembly shown in FIG. 3.

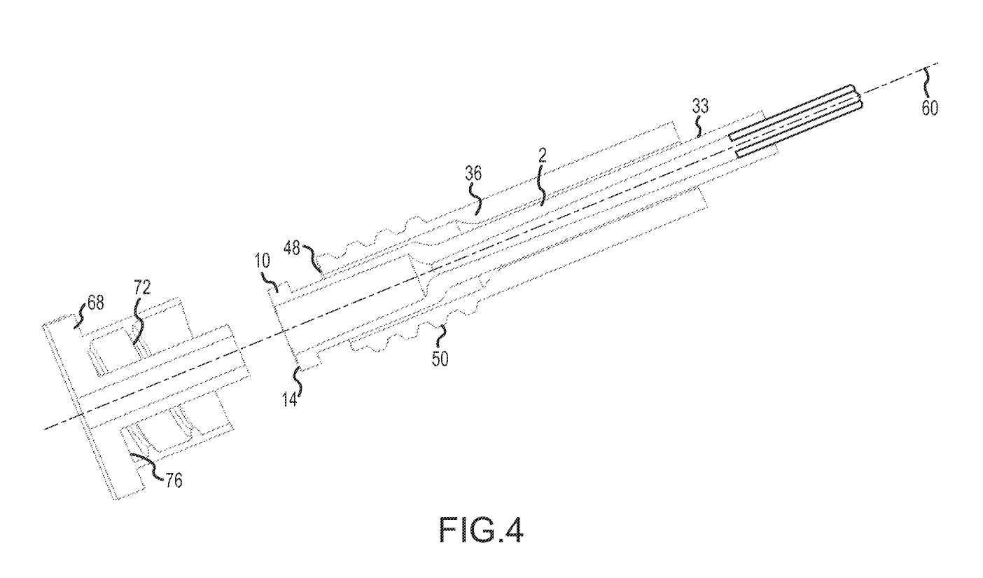

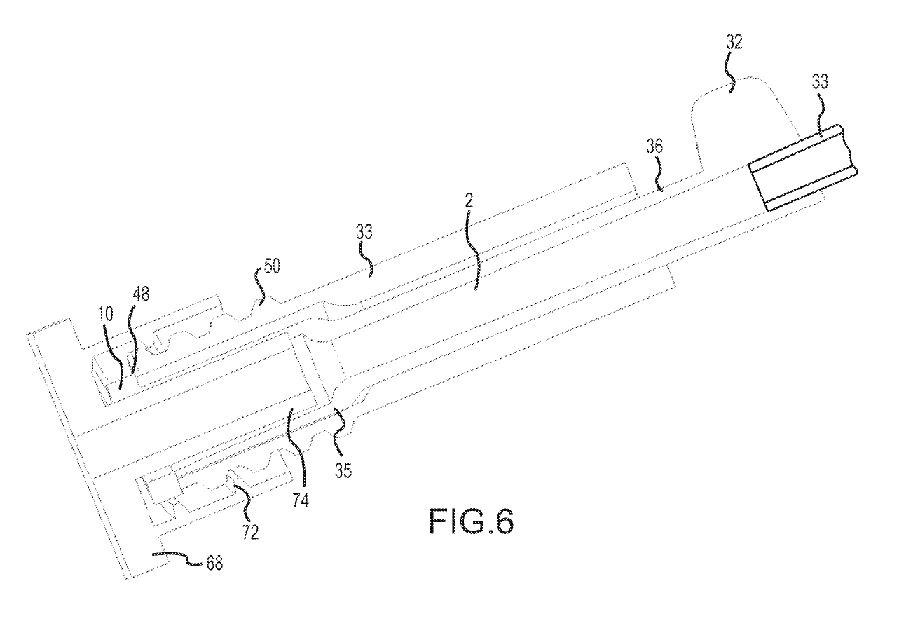

More specifically, FIGS. 3-7 show the female luer connector 2 that is inserted through the floating nut 36 until the annular flange 10 extends outside the first end 48 of the floating nut 36 and the fin 32 extends outside of the slot 44 to form the female luer connector assembly 64. The female luer 2 and floating nut 36 are able to rotate relative to each other about their shared longitudinal axis 60. The assembly 64 is inserted into the male-luer connector 68 of the capnograph and the floating nut 36 is rotated to tighten the threads 50 onto complementary threads 72 of the male luer connector 68. Before the floating nut 36 is completely tightened, and while the female luer 2 is still able to rotate inside the floating nut 36, the fin 32 is moved, i.e., rotated about the longitudinal axis 60, to position the reflective material 22 on the end face 18 of the annular flange 10 in such a way to correspond with the optical recognition system of the capnograph. Because the female luer connector of embodiments of the present invention do not have reflective material extending around the entire end face 18, the female luer 2 must be rotated until the proper orientation is acknowledged by the capnograph. Once the female luer 2 is properly oriented, the floating nut 36 is rotated to secure it to the male luer connector 68 of the capnograph. As the floating nut 36 is tightened to secure the female luer connector 2, the slot 44 will be narrowed which compresses the floating nut about a portion 35 of the female luer 2 to maintain the rotational orientation of the female luer.

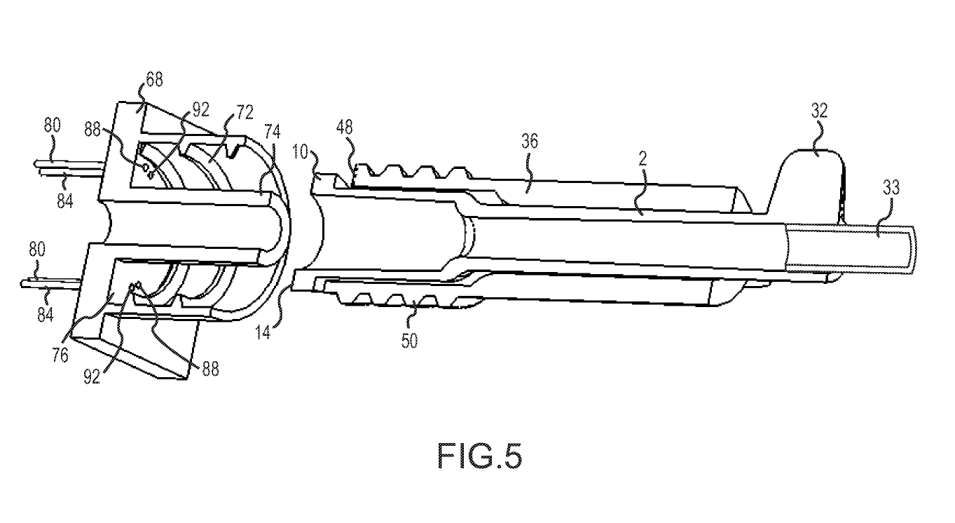

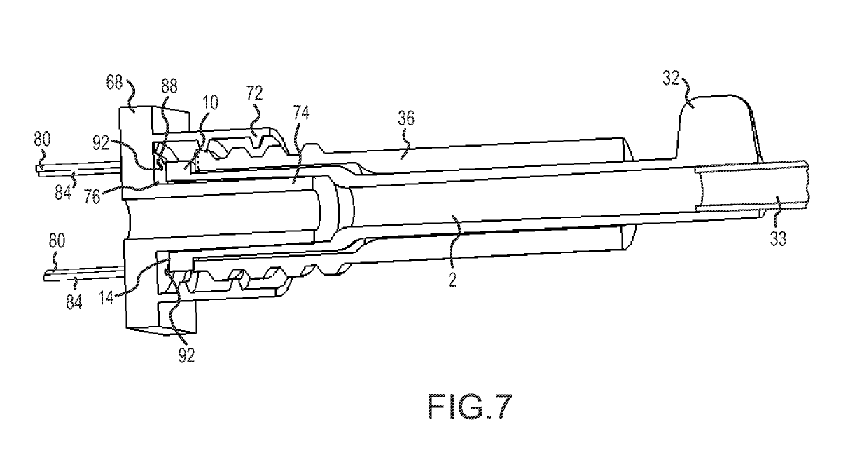

The male luer connector 68 includes a transmitter fiber optic cable 80 and a sensor fiber optic cable 84 that interconnects transmitting and sensing components within the medical instrument (not shown) to a transmitter optical opening 88 and a sensor optical opening 92 (see FIGS. 5 and 7). Although two transmitter/sensor pairs are shown, one of skill in the art will appreciate that a single pair or more than two pairs may be provided. Further, some medical instruments are programmed to receive signals from a first set of transmitters/receivers and not to receive additional signals. For example, the female luer may have multiple sensor areas, some of which require reflection of an optical signal and some that require no reflection. If multiple signals are received or if an unintended sensor receives a signal, the medical instrument will not function. Further, it is contemplated that the female luer's reflective areas may be dispersed or fixed in any pattern required by the medical instrument, provided the operator understands the necessary orientation, which is achieved by selectively rotating the fin.

In operation, a tubular extension 74 of the male luer connector 68 is inserted into the female luer connector 2. Rotation of the floating nut 36 interconnects threads 50 with threads 72 to secure the female luer connector 2 and the male luer connector 68. After the female luer 2 is interconnected to the male connector 68, the face 18 of the first end 14 of the female luer connector 2 is, in one embodiment, spaced from a face 76 of the male connector 68 such that openings 88 and 92 are spaced from the reflective portion of the female luer connector 2. To verify that an acceptable tube connector and the tube is interconnected to the medical instrument, optical signals are sent through the transmitter fiber optic cables 80, which exit via the transmitter optical openings 88, reflect off the reflective coating on the face 18 of the (see FIG. 3), and enter the sensor optical openings 92. The reflected signal(s) is sent via the sensor fiber optic cable 84 to a sensor associated with the medical device that confirms the existence of an acceptable tube connector. If, however, the reflective surface 22 (see FIG. 1) is oriented incorrectly and not positioned adjacent to the optical openings 88 and 92, the medical instrument will not function.

While various embodiments of the present invention have been described in detail, it is apparent that modifications and alterations of those embodiments will occur to those skilled in the art. However, it is to be expressly understood that such modifications and alterations are within the scope and spirit of the present invention, as set forth in the following claims. Further, the invention(s) described herein is capable of other embodiments and of being practiced or of being carried out in various ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

What is claimed is:

1. A female luer connector assembly comprising:
a first member having a first end with a flange extending therefrom, said flange having an end face, and a second end with a protrusion extending therefrom, said end face having a reflective material on less than the entire surface area thereof; and
a second member having a first threaded end which is adapted to be received within mating threads of a medical instrument, and a second end having at least one protruding tube, wherein said second member operably positioned around said first member and freely rotatable relative thereto; said second member having a longitudinal slot along a surface thereof for receiving said protrusion.

2. The connector assembly of claim 1, wherein said second end of said first member is adapted to receive a tube.

3. The connector assembly of claim 1, wherein said reflective material forms a predefined pattern.

4. The connector assembly of claim 1, wherein said protrusion is aligned with an orientation marking of the medical instrument when the connector assembly is interconnected to the medical instrument.

5. The connector assembly of claim 1, wherein said reflective material covers at least about 50% of said end face.

6. The connector assembly of claim 1, wherein said reflective material extends in a 180 degree arc of said end face.

7. The connector assembly of claim 1, wherein said first member includes an interior bore with a first inner diameter and a second inner diameter, said first inner diameter corresponding with said first end of said first member and being greater than said second inner diameter.

8. A luer connector assembly comprising:
a first member having a first end with an end face, and a second end;
a reflective material on less than the entire surface area of said end face; and
a second member positioned around said first member and freely rotatable relative thereto, the second member having a first end, which is adapted to be received within a medical instrument.

9. The connector assembly of claim 8, wherein said first end of said first member includes a flange extending therefrom.

10. The connector of claim 8, wherein said second member includes an outwardly-extending projection that extends from an outer surface thereof.

11. The connector of claim 8, wherein said second member includes a longitudinal slot for receiving said outwardly-extending protrusion.

12. The connector of claim 8, wherein in said first end of said second member includes a means for interconnection.

13. The connector of claim 12, wherein said means for interconnection comprises at least one of threads, a luer connector, and a bayonet fitting.

14. The connector of claim 8, wherein said second end of said second member includes at least one protrusion.

15. The connector of claim 8, wherein said second end of said first member is adapted to receive a tube.

16. The connector of claim 8, wherein said reflective material is a coating in a predefined pattern.

17. The connector of claim 8, wherein said at least 50% of said end face is covered by said reflective material.

18. The connector of claim 8, wherein said reflective material is positioned on at least about a 180 degree arc of said end face.

19. A connector for selective interconnection to a medical instrument, which has a male interface with a transmitter and a receiver, comprising:
a first member having a first end, a flange having an end face, and a second end;
a reflective material positioned on less than the entire surface area of said end face;
a second member having a first end that is interconnected to the medical instrument;
wherein said second member is positioned around said first member and freely rotatable relative thereto; and
wherein the transmitter emits a signal that reflects off said reflective surface to be received by said receiver to indicate that said connector is suited for the medical instrument

20. The combination of claim 19, wherein said second member includes a protrusion that extends from an outer surface thereof.

21. The combination of claim 20, wherein said second member includes a longitudinal slot for receiving said protrusion.

22. The combination of claim 19, wherein in said first end of said second member includes a means for interconnection.

23. The combination of claim 22, wherein said means for interconnection comprises at least one of threads, a luer connector, and a bayonet fitting.

24. The connector of claim 19, wherein said second end of said second member includes at least one tab.

25. The combination of claim 19 wherein said male interface has a face that includes openings associated with said receiver and said transmitter, said face being positioned away with said end face of said first member when said connector is interconnected to said medical instrument.
